(19) United States
(12) Patent Application Publication
(10) Pub. No.: US 2018/0256082 A1
(43) Pub. Date: Sep. 13, 2018

(54) METHOD AND DEVICE FOR NON-CONTACT SENSING OF VITAL SIGNS AND DIAGNOSTIC SIGNALS BY ELECTROMAGNETIC WAVES IN THE SUB TERAHERTZ BAND

(71) Applicant: NETEERA TECHNOLOGIES LTD., Jerusalem (IL)

(72) Inventors: Yochanan STEINBERG, Jerusalem (IL); Hanoch KISLEV, Zichron Yaakov (IL)

(21) Appl. No.: 15/861,721

(22) Filed: Jan. 4, 2018

Related U.S. Application Data
(60) Provisional application No. 62/470,256, filed on Mar. 12, 2017.
(60) Provisional application No. 62/470,259, filed on Mar. 12, 2017.

(51) Int. Cl. A61B 5/1455; A61B 5/024; A61B 5/0205

(52) U.S. Cl. CPC A61B 5/1455; A61B 5/11; A61B 5/0205; A61B 5/02405

(57) ABSTRACT

A system for non-invasively detecting vital signs of a subject, including a) a sub-THz beam source, b) an optical interferometer that is configured to accept the sub-THz beam, split the sub-THz beam into a reference beam and a measurement beam, focus the measurement beam onto a subject, accept a reflection of the beam from the subject and combine the reflection of the measurement beam with the reference beam; c) a detector configured to detect the combined beam; and an electronic circuit configured to receive and analyze the detected combined beam and identify vital signs of the subject.

### RELATED APPLICATIONS

This application claims priority from provisional application No. 62/470,256 dated Mar. 12, 2017 and 62/470,259 dated Mar. 12, 2017, the disclosures of which are incorporated herein by reference.

### TECHNICAL FIELD

The present disclosure relates generally to contact free vital sign monitoring and motion classification of a living being.

### BACKGROUND

Currently, there are many technologies, both invasive and less invasive, e.g. contact based sensors that are used to obtain vital sign and diagnostic information of a person. Examples are various types and methods of thermometers to measure body temperature, commonly used contact based SpO₂ sensors that measure heart rate and oxygen levels, cuffed style brachial artery blood pressure devices, and multiple contact ECG systems to measure the electrical activity of the heart.

Vital sign data can be acquired for example by using piezo-electric based sensors or other types of contact based sensors that respond to mechanical action, such as tonometer based instruments that can measure the pulse pressure wave. All of these sensors can provide valuable diagnostic information regarding the person being measured. There are growing needs however, for the ability to acquire such information without having to physically place a sensor in, on, or even near a subject being tested or monitored. Consequently, this requires a means of obtaining such data using wireless technologies where the sensing device is physically separated from the person being measured. The information that is necessary to collect from a human to provide the type of vital sign data previously mentioned implies the ability to remotely detect thermal, electromagnetic, and acoustic, emissions and reflections as well as spatially and temporally sampling of surface and internal displacements or other periodic vibrations of a region or regions of the person being monitored.

The remote measurement of human vital signs requires the ability to temporally resolve the spatial displacements of a reflecting body surface where for example the impact of the mechanical action of the heart and lungs can be sensed. Those skilled in the art utilize various wireless techniques to obtain the time evolving spatial information. One method that is often used in the general field of radar are coherent Doppler based systems to extract quantities relating to the phase or computing the actual phase of the reflected signal to measure the spatial displacements of the reflecting surface. Another method to obtain the temporal phase information uses interferometric methods, commonly called the Michaelson interferometer. This method splits a collimated beam into two paths, one being the reference beam, and the second called the measurement beam. Upon reflection from the subject under test the measurement and reference beams are recombined at a beam splitter and propagate towards a suitable detector. The time varying intensity level of the recombined beam contains the temporally varying spatial displacement information due to the respiration and heart rate data of the person being monitored.

### SUMMARY

An aspect of an embodiment of the disclosure relates to a system and method for non-invasively detecting vital signs of a subject by using an optical interferometer to illuminate the subject with a sub-THz measurment beam and combine a reference beam with a reflection of the measurment beam from the subject. The combined beam is then detected by a detector and provided to an electronic circuit for analysis to identify vital signs of the subject.

The human body provides valuable information relating to the general state of health and important vital sign information regarding the condition of internal systems simply through the very small quasi-periodic displacements that can be observed on the body surface. The ability to observe these displacements using non-contact based technologies, wirelessly and remotely, and having see-through soft material capabilities, presents a new opportunity to quickly, accurately, and without interfering the test subject to obtain valuable information, both in monitoring and diagnostic situations.

An optically based solution to obtain spatial displacement information called a Michaelson interferometer is commonly used to accurately and precisely measure path length differences, to fractions of the wavelength of the source frequency being used. However these setups are typically large scale and inconvenient for use as a practical, portable handheld sensor.

In an exemplary embodiment of the disclosure, a miniaturized optical interferometer for mm waves is used for non-invasively detecting vital signs of the subject. Optionally, the miniaturized optical interferometers for mm waves may encounter degraded performance due to the need to actively isolate between the measurement beam and other beams within the interferometer resulting from multiple reflections from the internal walls of the interferometer during propagation. Upon reduction in size the system necessarily approaches waveguide-like scales, however the complexities of the geometry, routing, splitting, reflecting, and recombining beams may result in many unwanted reflections that can corrupt the detected recombined signals.

Optionally, the disclosed interferometer eliminates unwanted multiple reflected signals by treating the interior walls of the interferometer waveguide-like structure to ensure that the multiple reflections are minimized as the beams propagate along their respective paths. The treatment may include coating the inner walls or filing the inner walls to make them less smooth.

In an exemplary embodiment of the disclosure, quantitative vital sign data relating to the respiratory and cardiac functions of a human are obtained and provide important information for many home, commercial, and clinical applications. In addition, determination of a subject's particular state of movement is classifiable using this technology.

There is thus provided according to an exemplary embodiment of the disclosure, a system for non-invasively detecting vital signs of a subject, comprising:

A sub-THz beam source; An optical interferometer that is configured to accept the sub-THz beam, split the sub-THz beam into a reference beam and a measurement beam, focus the measurement beam onto a subject, accept a reflection of the beam from the subject and combine the reflection of the measurement beam with the reference beam;

A detector configured to detect the combined beam; and An electronic circuit configured to receive and analyze the detected combined beam and identify vital signs of the subject.

In an exemplary embodiment of the disclosure, the vital signs are selected from the group consisting of: respiration rate, heart rate, respiration and heart rate intervals and respiration and heart rate variabilities. Optionally, the source provides a beam with a frequency between 50 to 1000 GHz. In an exemplary embodiment of the disclosure, the interferometer includes at least one mirror and at least one beam splitter. Optionally, the interferometer includes two beam splitters. In an exemplary embodiment of the disclosure, the interferometer comprises the source and detector on the same side. In an exemplary embodiment of the disclosure, the source beam and combined beam form a primary plane and the measurement beam probes a subject on an axis perpendicular to the primary plane. Optionally, the interferometer includes inner walls that are coated with an absorbing material. In an exemplary embodiment of the disclosure, the interferometer includes inner walls that are treated to have surface features that eliminate the unwanted effects of multiple scattering and reflections of a sub-THz beam. Optionally, a motion sensor is coupled to the interferometer for considering motion of the interferometer when analyzing the detected combined beam. In an exemplary embodiment of the disclosure, a range finder is coupled to the interferometer for considering the distance between the interferometer and the subject when analyzing the detected combined beam. Optionally, the interferometer elements form a dish antenna collector structure. In an exemplary embodiment of the disclosure, the system comprises multiple interferometers configured to measure different locations on a subject simultaneously. Optionally, the multiple interferometers use different frequency sub-THz beams.

There is further provided according to an exemplary embodiment of the disclosure, a method of non-invasively detecting vital signs of a subject, comprising:

Transmitting a sub-THz beam from a beam source;

Receiving the sub-THz beam by an optical interferometer;

Splitting the sub-THz beam into a reference beam and a measurement beam;

Focusing the measurement beam onto a subject;

Accepting a reflection of the beam from the subject;

Combining the reflection of the measurement beam with the reference beam;

Detecting the combined beam by a detector;

Receiving and analyzing the detected combined beam by an electronic circuit; and

Identifying vital signs of the subject by the analyzing.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a perspective top view of an interferometer system for measuring a signal reflected from a subject, according to an exemplary embodiment of the disclosure;

FIG. 2 is a schematic illustration of a top view of an alternative interferometer system with the source and detector on the same side, according to an exemplary embodiment of the disclosure;

FIG. 3 is a schematic illustration of a top view of an alternative interferometer system that simultaneously measures the reflection and the optical interferometric information from the subject, according to an exemplary embodiment of the disclosure;

FIG. 4 is a schematic illustration of a side view of a dish antenna collector structured interferometer, according to an exemplary embodiment of the disclosure;

FIG. 5 is a schematic illustration of a side view of a center feed dish antenna collector structured interferometer, according to an exemplary embodiment of the disclosure;

FIG. 6 is a schematic illustration of a perspective top view of an interferometer system for measuring a signal reflected from a subject that probes a subject on an axis perpendicular to a primary plane of the interferometer, according to an exemplary embodiment of the disclosure;

FIG. 7 is a schematic illustration of deploying a system for measuring a signal reflected from a subject, according to an exemplary embodiment of the disclosure;

FIG. 8 is a schematic illustration of a perspective top view of an interferometer system for measuring a signal reflected from a subject that probes a subject on an axis perpendicular to a primary plane of the interferometer, according to an exemplary embodiment of the disclosure;

FIG. 9 is a graph of an intensity signal using an interferometer measuring a human subject, according to an exemplary embodiment of the disclosure;

FIG. 10 is a graph of a Ballistocardiogram (BCG) measured with a sub-THz interferometer, according to an exemplary embodiment of the disclosure;

FIG. 11a is a graph of a Ballistocardiogram (BCG) measured from a subject through the back of a chair, according to an exemplary embodiment of the disclosure;

FIG. 11b is a graph of a heart rate interval computed from a pulse wave, according to an exemplary embodiment of the disclosure.

### DETAILED DESCRIPTION

In an exemplary embodiment of the disclosure, a sub-THz signal/beam is used to measure vital signs of a subject. The vital signs may include breathing rate, heart rate, pulse rate and other parameters. Optionally, use of a sub-THz signal/beam having a small wavelength provides enhanced accuracy. The measurements are received using an optical interferometer (generally known as a Michaelson interferometer) comparing/combining a reference signal to a resulting test signal, wherein the interferometer interacts with the sub-THz signal using optical characteristics, such as reflection, refraction, focusing and absorbing to form a combined signal in contrast to using electrical characteristics such as by using antennas to accept the sub-THz signals. Optionally, the sub-THz measurements may be used in combination with measurements from other sensors (e.g. a sensor measuring subject/interferometer motion or subject/interferometer relative position).

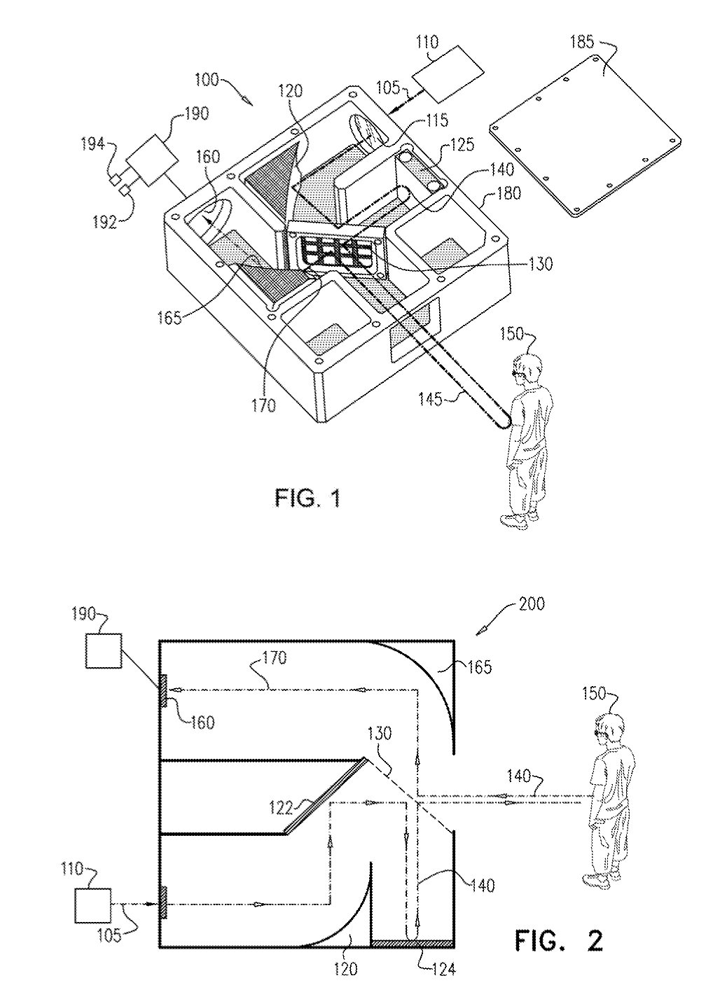

FIG. 1 is a schematic illustration of a perspective top view of an interferometer system 100 for measuring a signal 145 reflected from a subject 150, according to an exemplary embodiment of the disclosure. In an exemplary embodiment of the disclosure, a terahertz source 110 (e.g. produced by a high gain antenna) emits a terahertz beam 105 directed toward a collimating mirror 120. The collimating mirror 120 reflects the beam 105 toward a beam splitter 130. Optionally, the beam 105 is split into a reference beam 140 and a measurement beam 145. The reference beam 140 travels to a flat plate mirror/reflector 125 that reflects the reference beam 140 back toward splitter 130. The measurement beam 145 travels out from the interferometer 100 toward the subject 150 and is reflected back toward splitter 130. In an exemplary embodiment of the disclosure, the reflected measurement beam 145 and the reflected reference beam 140 are recombined at the splitter to form a recombined beam 170 that propagates toward a mirror 165 (e.g. an off axis reflecting mirror) that focuses the recombined beam 170 onto a detector 160.

In an exemplary embodiment of the disclosure, the interferometer 100 is designed for use with a sub-THz beam, e.g. having a frequency of between 1 GHz-1 THz or between 50 GHz to 1000 GHz or even 50 GHz to 500 GHz. Optionally, the components of the interferometer 100 are selected to interact optically with the sub-THz beam, the components include:

1. A mirror (e.g. 120, 165), for example a metallic material with a polished surface.

2. A beam splitter (e.g. 130), for example a thin metallic material with polished surfaces and a gridded/checker board like structure or pattern such that:
a. The area of the thin metallic area is approximately equal to the area of the open holes, per unit cell.
b. The area of the open holes is approximately 2-5 times the wavelength.
c. The pattern may be circular, square or randomly shaped.

3. An absorber (e.g. shown in FIG. 3), for example an artificial dielectric or resistive sheet absorber.

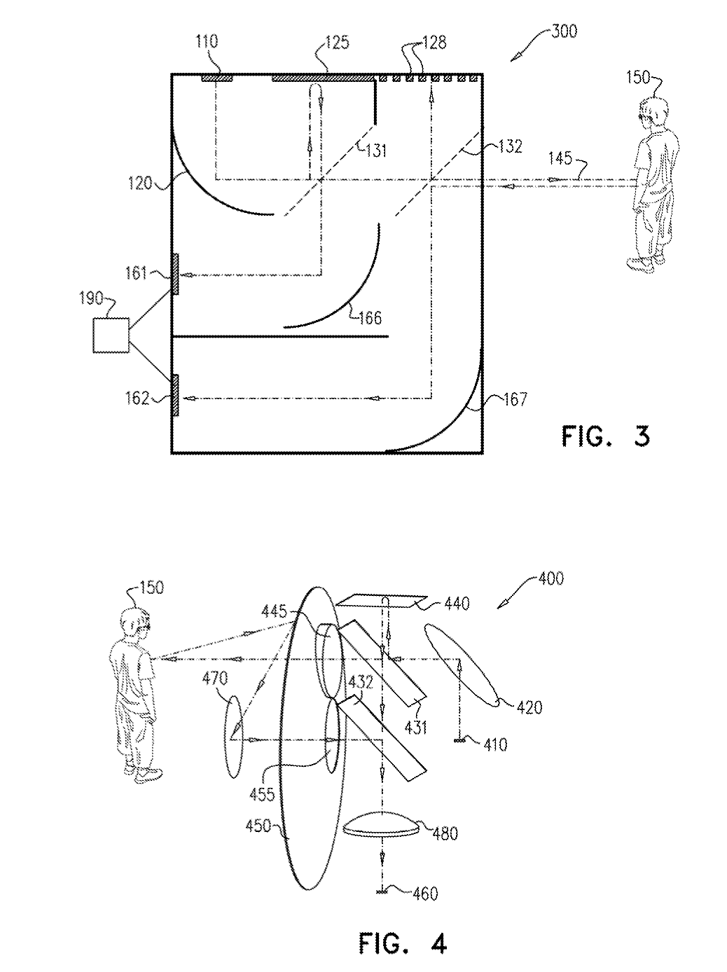

4. A lens (e.g. shown in FIG. 4), for example a custom built silicon lens that responds to sub-THz waves.

In an exemplary embodiment of the disclosure, the internal surfaces of the interferometer may be treated to reduce roughness and thus reduce internal reflections. Optionally, this could be done by physically modifying the structure of the walls (e.g. filing the surfaces to make them smoother) or by coating the surfaces with an absorbing material, or by a combination of mechanical means (e.g. filing, rubbing etc.) and coating with an absorbing material.

In an exemplary embodiment of the disclosure, sub-THz frequencies are used owing to their see-through soft material capability and sub-millimeter wavelengths that support detection of spatial displacements on order of fractions of these wavelengths. The current technique requires a coherent sub-THz source 110 and the ability to collimate the beam to a sufficient degree to ensure minimal beam divergence over the desired measurement range. The system further requires that the transmitted beam 105 be split into two paths, one a reference path and the second a measurement path. Upon reflection from the subject 150 under test the reflected measurement beam 145 is optically recombined with the reference beam 140 prior to detection forming recombined beam 170. The constructive and destructive interference between the reference and measurement beams provides to the detector variable intensity information that relates to the time varying spatial displacement of the reflecting surface being measured from subject 150, and can be mathematically described by the following:

I_{T}(t)=I₁+I₂+2\sqrt{I₁I₂} cos(2kΔx(t)+θₒ) (Eq. 1)

Where I_{T} is the total intensity measured at the detector, I₁ is the reference beam intensity, I₂ is the measurement beam intensity, k is the wavenumber, Δx(t) is the time dependent path length difference between the two beams and θₒ is the total residual phase term. The time dependence of the path length difference is the parameter that carries the information for vital sign monitoring. It can be expressed in the following way:

Δx(t)=g_{B}(t)+h_{H}(t)+n(t) (Eq. 2)

Where the functions g and h represent the time dependent displacements of the body surface due to the subject's breathing (B) and heart beat (H), respectively; n is a term that represents the cumulative effect of noise and all the other types of unwanted motion. The motion that is due to the respiration and heart rates of the test subject is typically quasi-periodic over the short time intervals and when the measurement area is taken from the chest or back areas, has distinctive temporal signatures that includes both respiration and heart rate information. Measurements taken from other areas of the body, such as the palms, limbs, or forehead often show a reduced respiratory signature but nevertheless contain important heart rate information.

The vital sign information is contained in the time varying path length variable Δx(t) and in Eq. 1 is embedded inside the cosine function. A Taylor series expansion about 9, of the cosine in Eq. 1 shows:

cos(2kΔx(t)+θₒ)≈2kΔx(t) when θₒ is an odd multiple of π/2. Eq. (3)

When Eq. (3) is satisfied the intensity in Eq. (1) is effectively a direct measurement of the temporal content of the spatial variations of the reflecting surface.

However, when the phase constant term θₒ is an even multiple of π/2 the Taylor series expansion shows:

cos(2kΔx(t)+θₒ)≈constant Eq. (4)

and in this condition the sensing pixel is in a dead spot, as such it does not detect the temporal variation of the reflecting surface, and the measured intensity remains constant to within the system noise.

In an exemplary embodiment of the disclosure, using a single pixel detector in an interferometric setup could be problematic due to the dead spot condition and may cause problems in implementing the above technique. However as described in U.S. patent application Ser. No. 15/636,667 dated Jun. 29, 2017 the disclosure of which is incorporated herein by reference, a CMOS based sub-THz detection system having a multi-pixel solution can be used, thereby escaping the dead spot pixel problem by a set of spatially distributed sensors. Optionally, the detector may use a 4×4 pixel layout with a 0.4 mm pixel pitch. This provides sufficient coverage of the interference pattern such that there always are several available pixels containing vital sign information at any given moment. The current algorithms track each pixel to determine which channel contains the highest SNR in terms of respiration and heart rate information.

In an exemplary embodiment of the disclosure, the respiratory and heart rate data can be computed with minimal processing, depending upon the data fidelity at any given moment. For example, during instances when there are increased noise levels, a short time Fourier transform technique can be used to compute a time averaged estimate of the desired signal, and in the frequency domain a typical respiratory signal is between 5-20 Bpm (breaths per minute), and a heart rate could be between 40-200 bpm (beats per minute). In this case however, additional steps are taken to avoid selecting the incorrect vital sign peak. Often, the correct spectral component does not necessarily have the largest spectral value. Therefore, smart filtering and processing techniques must be utilized to properly identify the fundamental component. When the noise level is lower however, the respiratory and heart rate data can be extracted in near real-time by using separate causal filters for the respiration and heart rate information. In other cases, such as when the measurements are made from the palm, the instantaneous heart rate can be read directly from the peak to peak interval in the raw data. The trace of the pulse wave by measuring the time dependent surface displacement is also known as a Ballistocardiogram (BCG) and contains information relating to the mechanical function of the heart. In situations when this information is available, usually corresponding to high vital sign SNR, the peak to peak interval is readily apparent, as seen for example in graph 1000 in FIG. 10, and easily computed. This type of information provides the heart rate interval and is necessary for computing the heart rate variability, which is becoming an increasingly important parameter for many different industries and applications.

In an exemplary embodiment of the disclosure, different realizations of possible interferometers (see FIG. 1 to FIG. 6 and FIG. 8) that could be used to acquire vital sign information. It should be noted that other designs may be implemented to serve as interferometers.

In an exemplary embodiment of the disclosure, interferometer 100 is fabricated using a rectangular body 180 (e.g. made from aluminum or other metals) with multiple channels for guiding the reference beam 140, measurement beam 145 and recombined beam 170. Beam splitter 130 is installed at the crossing of the channels in order to split the beam transmitted from the source 110 into the measurement beam 145 and reference beam 140. The measurement beam 145 reflected from the subject 150 is optically coupled with the reference beam 140 to recombined beam 170 which is focused by mirror 165 onto the detector 160. Optionally, the detector 160 processes the recombined beam and feeds an electrical signal to an electrical circuit 190 to extract the phase information. Optionally, electrical circuit 190 may include a processor and memory (or may be a general computer) which includes an application to analyze information received from detector 160. In an exemplary embodiment of the disclosure, the body 180 is covered with a suitable cover 185 (e.g. a metal cover) which shields the interferometer 100 and prevents the entrance of external radiation towards the opening of the detector 160.

In an exemplary embodiment of the disclosure, for a 400 GHz center transmitter frequency, the cross section of the channels is optionally selected as 25 mm (width) by 30 mm (deep). The surface roughness is characterized by a generalized 0.2 mm spatial period and 0.15 mm peak to valley depth. The focusing reflectors (120, 165) adjacent to the source 110 and detector 160 are off-axis parabolas with a typical effective focal length of 75 mm. The selected parameters are scaled according to the particular frequency used.

In an exemplary embodiment of the disclosure, systematic biases, or false readings due to internal reflections are eliminated due to the disclosed controlled surface roughness that are an integral part of the absorbing wall waveguide structure. The surface roughness may be periodic or non-periodic, ordered or random in their structure, dimensions, and patterns. The integral property of this feature being the ability to allow the central region of the beam to propagate through the system with minimal power density loss while eliminating reflections and scattering at the edges of the channels edges that would contribute to and possibly saturate the nature of the recombined measurement beam 140 and reference beam 145.

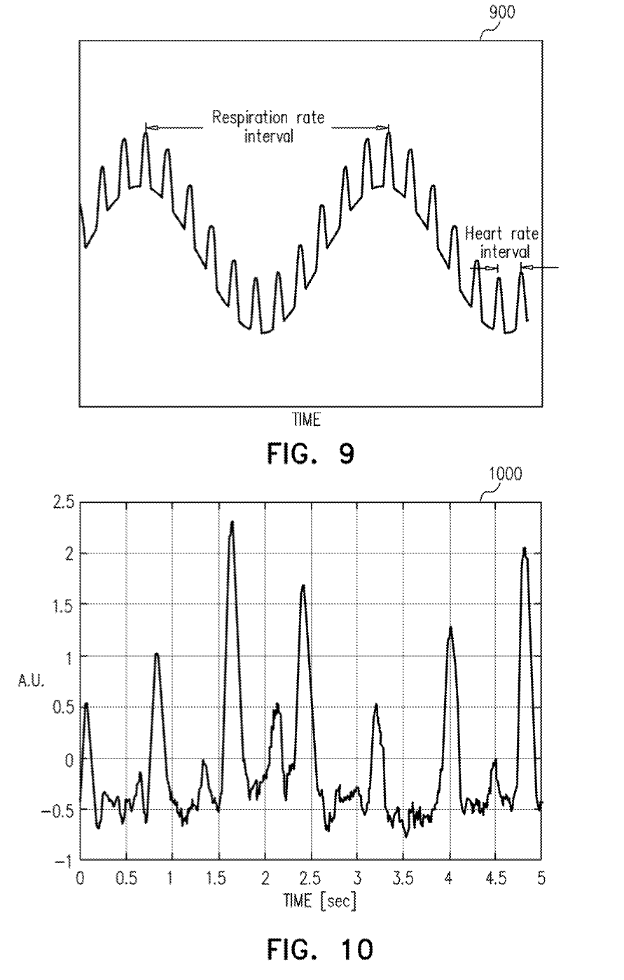

In an exemplary embodiment of the disclosure, detector 160 measures the intensity of the recombined beam 170 whose signal level varies for example as shown in graph 900 in FIG. 9. When the reflection is collected from the chest area of the subject 150 for example, the time varying information contains both heart rate, respiration rate, and heart rate interval information. The data is extracted by computing the time intervals between local short term peaks to obtain the heart rate data and the longer interval peaks that contain the respiratory data. Alternatively, these data are obtained by utilizing short term Fourier transform techniques as previously described.

FIG. 9 represents the expected signal level from a subject 150 under test using an interferometric setup. The lower frequency signal represents the longer time scale respiration rate, the higher frequency signal represents the shorter time scale heart rate.

FIG. 10 is an example of a BCG measured from the back of a human sitting in a chair using a sub-THz based interferometer. The fidelity of the data allows beat by beat determination of the heart rate and thus the heart rate interval. The data further illustrates one of the unique features of using sub-THz energy, namely the ability to see through soft materials, an office chair and a fully clothed person in this case, and extract physiological data from the subject 150 being measured. A calibrated measurement of this signal could yield the pulse pressure, this in turn can be utilized to compute the pulse wave amplification which serves as a biomarker for cardiovascular disease.

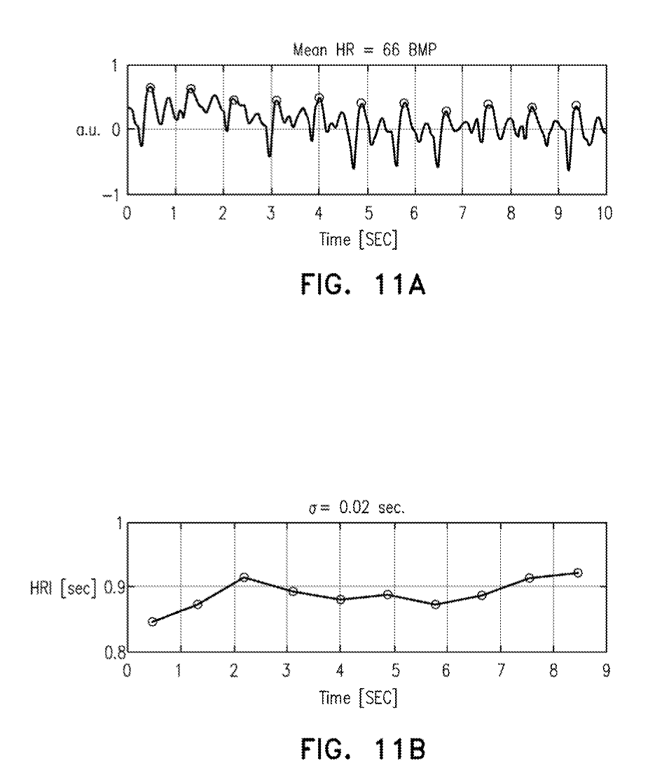

FIG. 11(a) is another example of a BCG measured through the back of a chair 710 from a subject 150 sitting in the chair 710. FIG. 11b shows the heart rate interval for the data collected in (a).

In an exemplary embodiment of the disclosure, the sub-THz frequencies used are capable of propagating through many common types of soft materials and non-conducting materials, such as plastics, fabrics, and clothing, however are reflected from the skin of a human so that they are returned back to interferometer 100 to determine physiological information from the surface displacement of the human surface measured over time. The sub-THz wavelengths are sensitive to small surface displacements of a human due to naturally occurring phenomena such as respiration and heart rate pulsations that can be inferred from time varying surface variability.

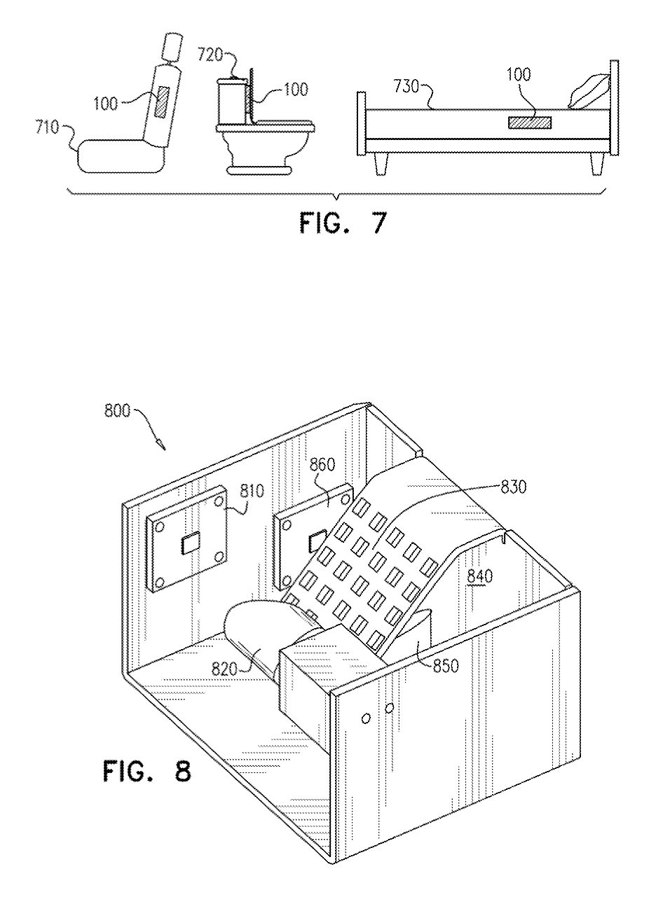

In an exemplary embodiment of the disclosure, the frequencies of the transmitted energy may range from 1 GHz up to 1 THz or even from 50 GHz to 1000 GHz or 50 GHz to 500 GHz. Exemplary applications using interferometer 100 include serving as a stand-alone sensor that can be directed at a subject of interest, automatically tracking the subject; or used as embedded sensors inside furniture (e.g. as shown in FIG. 7) such as installing interferometer 100 (the sensor) in tables, seats 720, chairs 710, beds 730, in accessories such as lamp shades, inside structures such as inside walls or other locations. The sensor could be utilized inside vehicles, at home or in health care settings to provide an indication related to the presence of a subject or status of a subject based on vital signals.

In an exemplary embodiment of the disclosure, interferometer system 100 has a motion sensor 192 and/or a range finder 194 coupled to it internally or externally (e.g. inside body 180 or coupled to the body externally). Optionally, information from the motion sensor 192 and/or range finder 194 can be used by electronic circuit 190 to analyze the information from the interferometer 100, for example to compensate for motion of the interferometer 100 relative to subject 150.

The interferometer system 100 could also utilize simultaneously or intermittently different frequencies, for example a lower frequency such as 50 GHz to measure large scale motions and 200 GHz to measure smaller spatial displacements of the subject being measured. The solution could also utilize more than one interferometer systems 100 having a single-frequency or with multiple-frequencies (each interferometer 100 using a different frequency) spatially distributed around a subject to extract vital sign information.

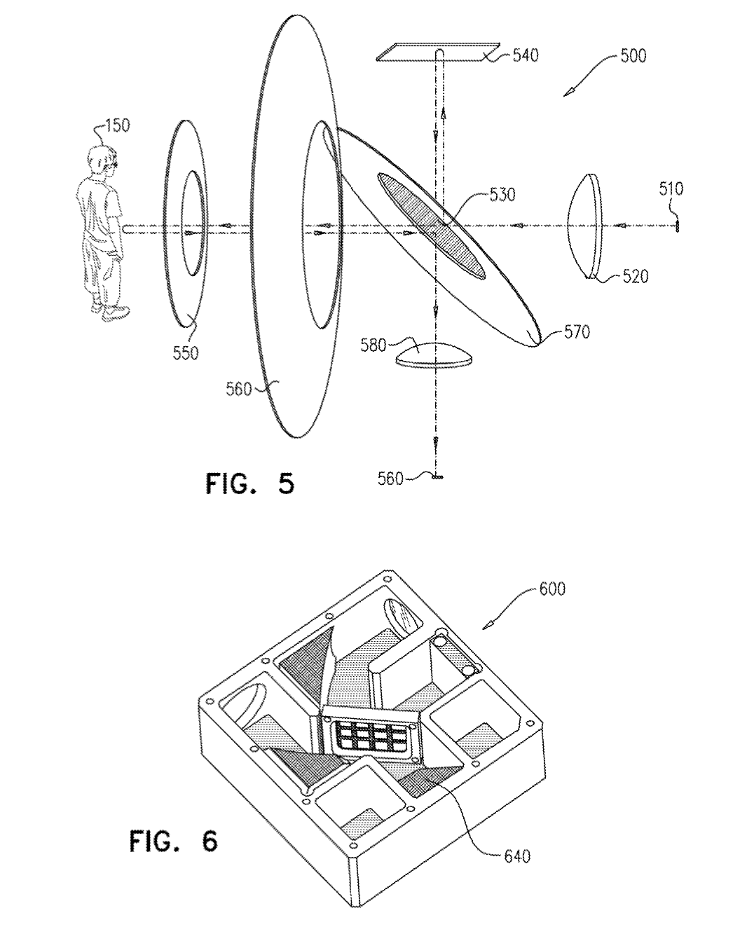

In an exemplary embodiment of the disclosure, interferometer 100 is constructed as a monolithic device with mirrors, beam splitter, or lenses serving as a part of the outer structure of the system, for example in the form of a dish antenna as shown in FIGS. 4-5. Alternatively, interferometer 100 may be formed as chip solution that would provide a much smaller footprint and still provide the same type of information necessary for vital sign monitoring, for example as shown in FIG. 8.

FIG. 2 is a schematic illustration of a top view of an alternative interferometer system 200 with the source 110 and detector 160 on the same side, according to an exemplary embodiment of the disclosure. This embodiment includes a reflecting mirror 122 and a reference mirror 124 to enable placing the source 110 and the detector 160 on the same side of the interferometer 200 and examining a subject 150 with the measurement beam 145 emitted from the other side of the interferometer 200.

FIG. 3 is a schematic illustration of a top view of an alternative interferometer system 300 that simultaneously measures the reflection and the optical interferometric information from the subject 150, according to an exemplary embodiment of the disclosure. Interferometer 300 includes two beam splitters 131 and 132. The first one combining the reference beam 140 with the reflected measurement beam 145, wherein the reference beam 140 propagates through mirror 125, a focus mirror 166 and then to a first detector 161 combined with the reflected measurement beam 145. The second splitter 131 with the help of an absorber 128 and a focus mirror 167 sends the reflected measurement beam 145 to the second detector 162 without the reference beam 140. Optionally, electronic circuit 190 can then use the additional information (from the two detectors (161, 162) to enhance analysis of the state of the subject 150. Optionally, the direct measurement of the reflected beam 145 in addition to the combined beam provides additional information relating to the reflection coefficient of the measured object and allows for example the ability to distinguish between an empty chair or an occupied chair by an inanimate object such as a package, or by a human body without a discernable heartbeat.

FIG. 4 is a schematic illustration of a side view of an off axis transmit beam dish antenna collector structured interferometer 400, according to an exemplary embodiment of the disclosure. Optionally, interferometer 400 includes a sub-THz source 410, an off-axis parabolic mirror 420, a first beam splitter 431, a reference mirror 440, a hole 445, a parabolic dish 450 (e.g. about 100 mm-150 mm), a hole 455 in dish 450, a dish feeder reflector 470, a second beam splitter 432, a focusing lens 480 (e.g. a 30 mm in diameter aspheric lens), and a detector 460 (e.g. a 4×4 array detector).

In an exemplary embodiment of the disclosure, the subject 150 is located to the left of parabolic dish 450, the beam exits at hole 445, the parabolic dish 450 collects the reflected energy from the subject 150 and focuses it onto the reflector mirror 470, which then routes the reflected beam to recombine with the reference beam at beam splitter 432.

Optionally, interferometer 400 functions in a similar manner as interferometer 100 except that it is generally more spread out in three dimensions and requires a larger volume to accommodate it.

FIG. 5 is a schematic illustration of a side view of a center feed dish antenna collector structured interferometer 500, according to an exemplary embodiment of the disclosure. Optionally, interferometer 500 includes a sub-THz source 510, a collimating lens 520, a beam splitter 530, a reference mirror 540, a dish feeder reflector 550 (the subject stands to the left of the dish 550), a parabolic dish 560, an off-axis parabolic mirror 570, an aspheric lens 580, a detector 560 (e.g. a 4×4 array detector). Optionally, interferometer 500 functions in a similar manner as interferometer 100 except that it is generally more spread out in three dimensions and requires a larger volume to accommodate it. Additionally, this particular design allows for symmetrical reception of the reflected beam over a larger area, allowing for greater target alignment capability. The waves returned on axis are optically merged with the reference beam at the beam splitter 530. The waves intercepted by the dish 550 are focused around the aspheric lens 580 and coupled with the reference beam at a detector 560.

FIG. 6 is a schematic illustration of a perspective top view of an interferometer system 600 for measuring a beam reflected from a subject 150 that probes a subject on an axis perpendicular to a primary plane of the interferometer, according to an exemplary embodiment of the disclosure. Interferometer 600 is similar to interferometer 100, except that the collimated beam is reflected by a deflecting mirror 640 to a direction perpendicular to the main sensor package plane. This allows for placing the interferometer system 600 embedded within certain furniture and platforms that require a small width footprint. Optionally, other embodiments could accomplish the same objective of having the measurement beam reflected in a direction perpendicular to the primary optical plane.

FIG. 8 is a schematic illustration of a perspective top view of an interferometer system 800 for measuring a beam reflected from a subject 150 that probes the subject 150 on an axis perpendicular to a primary plane of the interferometer 800, according to an exemplary embodiment of the disclosure. Interferometer 800 is a compact interferometer design that has the source and detector located on the same plane and a measurement beam redirected in a direction perpendicular to the original optical plane. Interferometer 810 includes a source 810, an off-axis collimating mirror 820, a beam splitter 830, a reference mirror 840, a focusing mirror 850 and a multi-pixel detector 860.

In some embodiments of the disclosure, the optical mixing with an interferometer 100 that provides a fluctuating intensity level at the detector 160 could be replaced by an RF based solution that results in extraction of the phase information relating to the body surface displacements directly from the reflected RF signal, with a minimal amount of additional internal processing.

This RF based solution could be obtained by mixing the reflected signal with the original transmitted signal and converting the received signal to baseband. The baseband output obtained by this technique could be then processed in the same manner as already described for the vital sign data obtained by an interferometer.

The RF solution could also involve heterodyne processing to obtain the in-phase and quadrature components of the received signal and utilize arctangent demodulation to obtain the phase of the received signal. Using this type of data, the vital sign monitoring data processing would proceed as previously described.

The RF solution could involve using a select set or FMCW signals such that the carrier frequency is varied near the center frequency to address the issue of dead spots in the demodulated phase.

However it should be noted that the optical solution may improve the ability (e.g. timing and accuracy) to extract certain information and improve the ability to perform certain applications.

In an exemplary embodiment of the disclosure, several parameters and applications can be determined simply by monitoring the small surface displacements on the skin from a human. When the area of measurement is on or near the torso area of a human, and due to its large surface displacement, usually the primary signal observed is the respiration rate. Because of the expansion and contraction of the lungs as a person inhales and exhales, the chest area will rise and fall accordingly, at a quasi-periodic rate, usually between 10-20 breaths per minute for an adult.

A secondary signal, usually of smaller amplitude and at a higher frequency is the person's heart rate. The surface pulsations due to heart rate, can be observed in many places on a human body. The typically heart rates vary between 50-100 beats per minute for most people. This is at a noticeably higher frequency than the typical respiration rate, and usually having a smaller surface displacement.

In low signal to noise ratio cases where the fidelity of the pulse wave seen in FIG. 10 is not clearly observable, there typically remains sufficient spectral information within the data that allows for Fourier domain determination of the respiration and heart rates.

When there is sufficient signal to noise in the pulse wave additional information can be inferred from the subject being monitored. As a first example, a clearly defined pulse wave allows for the determination of the heart rate interval. Much like an R-R interval from a person being monitored in hospital setting with a typical ECG instrument. This heart rate interval provides a fundamentally important parameter relating to a person's physiological state and general well-being, namely the heart rate variability. One expected usage of this information is in a smart seat sensor for the automobile industry to monitor a driver's physiological parameters, and particularly his probability of falling asleep at the wheel while driving. The heart rate variability is an important metric in detecting this possible outcome. Sleep research has shown that this metric acts an early warning signal predicting the onset of sleep 1-2 minutes prior to the event. An early warning sleep detection system brings considerable added value to the ever-increasing number of safety features that automobile manufacturers are bringing to market.

An early warning smart seat sensor for detecting the onset of sleep for the automotive industry is just one example where such technology could be used. There are considerable number of other examples where such information brings considerable value and safety. For example, operators of heavy equipment or positions that require repetitive tasks, or jobs that require a human-in-the-loop with mundane activities, yet whose responsibilities have critical outcomes in terms of security or cost.

Another application of remote contactless pulse wave monitoring relates to general health awareness. For example, two adjacent measurements of the pulse wave provide a measure of the pulse wave velocity which is an indicator of arterial stiffness and correlates with cardiovascular disease.

Optionally, using a calibrated pulse wave monitor, further information can be collected from skin surface displacements, namely the blood pressure. Typically, non-invasive measurements of blood pressure are made using cuff-type sensors wrapped around the upper arm and known as the brachial artery blood pressure. Measurements can be made at other locations, the wrist for example, and yield a radial blood pressure. A sensor that calibrates the pulse wave to a spatial displacement could be further calibrated to provide a local pulse pressure similar to arterial tonometry.

Additionally, due to the high-fidelity nature of the pulse wave data, further diagnostic utilization of the pulse pressure could be made to infer the pulse wave amplification of a patient which is a biomarker of cardiovascular disease.

The high-fidelity pulse wave data contains not only the information previously mentioned, but the patterns and shapes of the pulse wave also relate to the electrical and mechanical operation of the heart. Information relating to the arterial tone of the artery at the skin level can be inferred by the pulse wave measurement.

Another application relates to the ability to extract from modulated displacements sounds in general and human speech in particular. By simultaneously sampling spatially diverse regions from a human, additional information can be inferred regarding the respiratory chest wall and abdominal movement paradox.

All of the above applications utilize remote and contact free sensing of a person's skin displacement, spatially and/or temporally. And due to the see-through properties of sub-THz waves, smart sensing platforms can form the critical infrastructure to many safety conscious technologies and industries.

In an exemplary embodiment of the disclosure, when test subjects are measured in a seat for example, there are often instances when motion of various types complicate the computation or interpretation of the vital sign information. Interestingly, the features in the data retain unique patterns that relate to the type of motion the test subject is undergoing. This immediately lends itself to the ability to classify the type of motion being experienced by the subject. For example, the subject could be speaking, or head turning, or arm movement, or leg movement, etc. and each of these conditions could be properly classified from each other, providing additional important information for a given monitoring situation.

In an exemplary embodiment of the disclosure, many applications are supported by use of an optical interferometer 100 or in other variations as described above. The applications include:
1. Non-contact based extraction of information from a human while in motion or at rest of the subject's heart rate data either computed in directly in the time domain, or in the Fourier domain, or by additional processing and cross correlating with a mathematical model.
2. Non-contact based extraction of information from a human while in motion or at rest of the subject's respiration rate data either computed in directly in the time domain, or in the Fourier domain, or by additional processing and cross correlating with a mathematical model.
3. Non-contact based extraction of information from a human while in motion or at rest of the subject's respiration rate interval either computed in directly in the time domain, or by additional processing and cross correlating with a mathematical model.
4. Non-contact based extraction of information from a human while in motion or at rest of the subject's heart rate interval either computed in directly in the time domain, or by additional processing and cross correlating with a mathematical model.
5. By using non-contact based extraction of information from a human while in motion or at rest of the subject's respiration rate interval over time provides the basis to monitor the respiration rate variability.
6. By using non-contact based extraction of information from a human while in motion or at rest of the subject's heart rate interval over time provides the basis to monitor the heart rate variability.
7. Non-contact based extraction of information from a human of the subject's pulse wave velocity measured simultaneously at more than one location on the test subject.
8. Non-contact based extraction of information from a human of the subject's pulse wave velocity measured in a spatially continuous fashion along a region on test subject.
9. Non-contact based extraction of information from a human of the subject's pulse wave measured directly in the time domain.
10. Non-contact based extraction of information from a human of the subject's pulse pressure measured indirectly from calibrated time domain pulse wave data.
11. Non-contact based extraction of information from a human of the subject's pressure wave amplification directly in the time domain.
12. Non-contact based extraction of information from a human due to temporally and spatially varying displacements of the surface and subsurface features of a human.
13. Non-contact based classification of various states or types of human motion as commonly encountered in a given environment. Types of motion classification for a driver include but not limited to: head motion, arm motion, leg motion, torso movements, talking, sneezing and coughing and various types of motion due to forces external to the subject. For an environment of patient monitoring the motion classification would include in addition to the previously mentioned states, positional movements, turning, sitting upright, and lying down.
14. Non-contact based determination of a person's state of drowsiness.
15. Non-contact based determination of a person's general well-being.
16. Non-contact based determination of a person's BCG
17. Non-contact based determination of a person's emotional/mental states

The terms ‘processor’ or ‘computer’, or system thereof, are used herein as ordinary context of the art, such as a general purpose processor, or a portable device such as a smart phone or a tablet computer, or a micro-processor, or a RISC processor, or a DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or electronic circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry. The processor and program may constitute the same apparatus, at least partially, such as an array of electronic gates, such as FPGA or ASIC, designed to perform a programmed sequence of operations, optionally comprising or linked with a processor or other circuitry.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure. It will also be appreciated by persons skilled in the art that the present disclosure is not limited to what has been particularly shown and described hereinabove.

I/We claim:

1. A system for non-invasively detecting vital signs of a subject, comprising:
A sub-THz beam source;
an optical interferometer that is configured to accept the sub-THz beam, split the sub-THz beam into a reference beam and a measurement beam, focus the measurement beam onto a subject, accept a reflection of the beam from the subject and combine the reflection of the measurement beam with the reference beam;
a detector configured to detect the combined beam; and
an electronic circuit configured to receive and analyze the detected combined beam and identify vital signs of the subject.

2. A system according to claim 1, wherein the vital signs are selected from the group consisting of: respiration rate, heart rate, respiration and heart rate intervals and respiration and heart rate variabilities.

3. A system according to claim 1, wherein the source provides a beam with a frequency between 50 to 1000 GHz.

4. A system according to claim 1, wherein the interferometer includes at least one mirror and at least one beam splitter.

5. A system according to claim 1, wherein the interferometer includes two beam splitters.

6. A system according to claim 1, wherein the interferometer comprises the source and detector on the same side.

7. A system according to claim 1, wherein the source beam and combined beam form a primary plane and the measurement beam probes a subject on an axis perpendicular to the primary plane.

8. A system according to claim 1, wherein the interferometer includes inner walls that are coated with an absorbing material.

9. A system according to claim 1, wherein the interferometer includes inner walls that are treated to have surface features that eliminate the unwanted effects of multiple scattering and reflections of a sub-THz beam.

10. A system according to claim 1, wherein a motion sensor is coupled to the interferometer for considering motion of the interferometer when analyzing the detected combined beam.

11. A system according to claim 1, wherein a range finder is coupled to the interferometer for considering the distance between the interferometer and the subject when analyzing the detected combined beam.

12. A system according to claim 1, wherein the interferometer elements form a dish antenna collector structure.

13. A system according to claim 1, comprising multiple interferometers configured to measure different locations on a subject simultaneously.

14. A system according to claim 13, wherein the multiple interferometers use different frequency sub-THz beams.

15. A method of non-invasively detecting vital signs of a subject, comprising:
transmitting a sub-THz beam from a beam source;
receiving the sub-THz beam by an optical interferometer;
splitting the sub-THz beam into a reference beam and a measurement beam;
focusing the measurement beam onto a subject;
accepting a reflection of the beam from the subject;
combining the reflection of the measurement beam with the reference beam;
detecting the combined beam by a detector;
receiving and analyzing the detected combined beam by an electronic circuit; and
identifying vital signs of the subject by the analyzing.

16. A method according to claim 15, wherein the vital signs are selected from the group consisting of: respiration rate, heart rate, respiration and heart rate intervals and respiration and heart rate variabilities.

17. A method according to claim 15, wherein the source provides a beam with a frequency between 50 to 1000 GHz.

18. A method according to claim 15, wherein the interferometer includes at least one mirror and at least one beam splitter.

19. A method according to claim 15, wherein the interferometer comprises the source and detector on the same side.

20. A method according to claim 15, wherein the source beam and combined beam form a primary plane and the measurement beam probes a subject on an axis perpendicular to the primary plane.
